(19) United States
(12) Patent Application Publication
(10) Pub. No.: US 2022/0141230 A1
(43) Pub. Date: May 5, 2022

(54) MEASURING SYSTEM, COMMUNICATION COMPONENT, DEVICE, PROCESS AND COMPUTER PROGRAM FOR A COMMUNICATION COMPONENT OF A MEASURING SYSTEM FOR SYNCHRONIZING ACCESS DATA

(71) Applicant: Dräger Safety AG & Co. KGaA, Lübeck (DE)

(72) Inventors: Günter WAHLBRINK, Lübeck (DE); Jun FURUTA, Lübeck (DE); Hannes STURM, Lübeck (DE)

(21) Appl. No.: 17/516,328

(22) Filed: Nov. 1, 2021

(30) Foreign Application Priority Data
Nov. 2, 2020 (DE) 10 2020 128 744.5

(51) Int. Cl. H04L 29/06; H04Q 9/00; G06F 16/27

(52) U.S. Cl. CPC H04L 63/102; G06F 16/27; H04Q 9/00

(57) ABSTRACT

A measuring system (500), a communication component (200; 300a; 300b; 300c; 600; 700), a device (20), a process (10) and computer program of a communication component (200; 300a; 300b; 300c; 600; 700) of a measuring system (500) are provided. The measuring system (500) includes an additional communication component (200; 300a; 300b; 300c; 600; 700). The process (10) for the communication component (200; 300a; 300b, 300c; 600; 700) of the measuring system (500) includes management (11) of at least one personalized user with access rights in the measuring system (500) and storage (12) of access data for the at least one personalized user. Synchronization (13) of the access data of the at least one personalized user with the one or more additional communication components (200; 300a; 300b; 300c; 600; 700) is provided.

### CROSS REFERENCE TO RELATED APPLICATIONS

This application claims the benefit of priority under 35 U.S.C. § 119 of German Application 10 2020 128 744.5, filed Nov. 2, 2020, the entire contents of which are incorporated herein by reference.

### TECHNICAL FIELD

The present invention pertains to a measuring system, to a communication component, to a device, to a process and to a computer program for a communication component of a measuring system, especially but not exclusively to a concept for managing access data in a gas measuring system.

### TECHNICAL BACKGROUND

Measuring systems are built mostly from different components, which communicate with each other via different communication channels and over a plurality of hierarchical levels. Measuring systems are used for many different purposes especially in an industrial setting. Sensors are used, as a rule, directly at the location of the variable to be measured for checking or for controlling and regulating different process parameters, for example, temperature, pressure, flow or gas concentration. Transmitters process these measured signals in a suitable form and transmit these frequently to central analysis systems, for example, an MPC (memory-programmable control), to a regulator or to a controller, which makes possible a monitoring of even a plurality of measurement points, or a direct intervention in the process by a control at a central location, for example, in a control room. An increasing communication of these different components of a measuring system among each other or even with external systems such as higher-level memories and internet areas makes possible vast possibilities for checking, archiving, configuring and storing extensive system data of a measuring device even from non-central access points.

For example, an architecture of a measuring system may make provisions for one or more sensors each to be associated with a controller. Reference is made, as a rule, to a transmitter in the area of stationary gas detection technology. A sensor for measuring a gas concentration sends measured values to a transmitter, which transmits these measured values, after they have been processed and digitized in a suitable manner, to suitable receivers together with status information via a process interface.

A sensor is, for example, a measuring transducer, which may be a part of a measuring chain, and is configured for the conversion of physical and/or chemical measured variables (e.g., gas concentrations) into an electrical signal. It may contain additional elements, such as signal amplifiers, linearization features, standardization features, also in the form of complex digital processing devices. A transmitter may be configured for operating and for supplying a sensor or measuring transducer. It may also contain, for example, parts of the signal processing of the signals arriving from the sensor, unless these signal processing devices are already contained in the sensor. A transmitter may comprise an interface for maintenance and configuration, for example, a display. In addition, a transmitter may be configured for providing and transmitting usually standardized measured signals (e.g., 4-20 mA, 0-10 V, digital) plus optionally special states of the system, such as errors, warnings, exceeding of the measuring range, etc.

Reference can thus also be made below to the transmitter. A controller or MPC is here an implementation of an analysis and/or control unit. The term controller is therefore used to represent an implementation of an analysis and/or control unit. The plurality of controllers may then communicate with one another directly and/or they communicate with an additional management unit, which may, in turn, be at a hierarchically higher level than the controllers.

For example, the individual components of a gas detection system, which may be implemented mostly locally, separately from one another, can be protected from unauthorized access. In a complex measuring chain starting from a sensor to measured value-processing and -displaying, higher-level analysis and storage systems, there are a plurality of access points, all of which shall be protected from unauthorized access or manipulation, but they concern the same measuring chain.

This is usually done by entering a pin number or password in order to perform, for example, security-relevant adjustments or calibrations. In order to make a special access possible, different access rights are frequently provided. There are, for example, different (role-based) authorization levels for operators, maintenance staff or administrators, to whom different levels of influence (access authorizations) are assigned, for example, at a transmitter or sensor. In an analysis system, for example, a computer, there already are different physical access processes, which are embodied via the entry of passwords or also via processes that detect fingerprints or visual profiles of a user.

For example, different user management schemes are used in the components in conventional systems, e.g., at controllers and transmitters/sensors. These are role-based user management schemes and they differ mostly concerning the access rights, at least those of administrators compared to other users.

A uniform username/password can be used in some cases in applications with internet connection for cloud applications as well as for all corresponding terminals with “internet capability.” However, a global user management is not available for each device or possibly for any device in an industrial setting at the field level for various reasons (explosion protection, energy consumption). A direct cause of this is that the devices do not have a connectivity function at the field level, which is due, in turn, to the limited energy budget—due to low capacity of the processor interface or to explosion protection. Novel electronic components (e.g., controllers) can, however, make it possible to implement these functions in case of the same energy budget. Different user management schemes and possibly access right management schemes are therefore operated in the same system. This may mean a considerable effort depending on the extension and the implementation of a measuring system if, for example, access data or access rights are changed system-wide. Security aspects are likewise essential in this connection.

### SUMMARY

There is therefore a need against this background to create an improved concept for user management in measuring systems.

Exemplary embodiments are based on the core idea of introducing, for example, in a system with the architecture described in the introduction, a uniform, personalized and synchronized user management between the controller and the transmitter/sensor. It is recognized that a synchronization can take place between the controller and the transmitter/sensor or between a transmitter/sensor and a transmitter/sensor, so that the access data are standardized between the components that synchronize with one another. This can facilitate the user management especially in case of communication via digital field buses (e.g., 4-20 mA and HART (Highway Addressable Remote Transducer—a standardized communication system for building industrial field buses)).

Exemplary embodiments create a process for a communication component of a measuring system. The measuring system has one or more additional communication components. The process comprises a management of at least one personalized user with access rights in the measuring system (rights to access the measuring system) and storage of access data for the at least one personalized user. The process further comprises a synchronization of the access data of the at least one personalized user with the one or more additional communication components. The communication component comprises in this case a controller and the one or more additional communication components comprise one or more transmitters. The process comprises, in addition, the receipt of updated access data at the one or more transmitters or at another communication component communicating with the controller and a standardization of the updated access data among the one or more communication components. Exemplary embodiments may provide for an automated standardization even in case of updating of the access data.

The management of a personalized user profile makes possible a better adaptation of an access right profile to an individual user than would be possible in case of a role-based user management. Exemplary embodiments can thus achieve a standardization of the access data among the one or more communication components and thus save administrative effort, which would be necessary in case of separate user management processes. When, for example, a user changes user data (e.g., password), these can trigger corresponding changes on all other transmitters, controllers and possibly applications in the cloud or in a local IT (information technology) in an automated manner and they do not need to be carried out on all individual devices in a time-consuming manner. A user with global access rights, for example, an administrator, does not accordingly need to manage many different passwords any longer, because an identical access code, which is valid for the measuring system, is sufficient in this case corresponding to the user's access authorization level.

A user management can be carried out in a personalized manner in exemplary embodiments. On the whole, the security can be increased in exemplary embodiments, because user data do not have to be handled globally with a great effort any longer and rapid changes can be carried out when needed. Who has made changes (e.g., configuration), what changes the user has made and at what time can be reconstructed by a corresponding documentation at the user level.

The measuring system may comprise, for example, a gas measuring system, so that a corresponding personalized user management is implemented in the gas measuring system. The measuring system may comprise a gas detection system, so that corresponding detection components can also be synchronized within the framework of the above-described process. Here and below, a separation of gas measuring system and gas detection system shall be seen less technically than rather in terms of linguistic usage. For example, the triggering of different events or thresholds may be of greater interest in a gas detection system compared to the knowledge of a measured signal.

In some other exemplary embodiments, the synchronization may further comprise a synchronization of the access rights of the at least one personalized user. Exemplary embodiments can also make it possible to standardize the access rights.

The access rights may be based on a role assigned to the at least one personalized user. For example, role-based basic right profiles can facilitate the creation of an access right profile for a (new) user.

The measuring system may comprise in exemplary embodiments at least one controller and one or more transmitters communicating digitally with the controller, the transmitters being coupled with gas measuring sensors. Exemplary embodiments make it possible to synchronize personalized access data between controllers and transmitters.

The synchronization may be carried out at least partially via a digital field bus. Exemplary embodiments can make possible a communication of access data or changes thereof even via digital field buses.

Documentation of changes of the access data can be carried out in some exemplary embodiments, so that reconstruction of a change history can be made possible.

The communication component may comprise, for example, a controller and the one or more additional communication components may comprise one or more transmitters. The synchronization of the access data can then comprise a standardization of the access data at the plurality of transmitters by the controller. The coordination of the synchronization may be carried out by the controller in some exemplary embodiments.

The updated access data can be obtained at the controller, at a transmitter or at another communication component communicating with the controller. Exemplary embodiment can thus make it possible to receive updated access data from a plurality of access points of the measuring system or even from all access points of the measuring system. The other communication component communicating with the controller may comprise, for example, a control room in an IT system, which is a local IT system for the measuring system, or an access node in a cloud.

At least some exemplary embodiments may comprise a standardization of the updated access data at all communication components suitable for a user access. An automated system-wide synchronization/updating of the access data may thus possibly be achieved.

The process may comprise, furthermore, a management of personalized access rights for the at least one personalized user. Some exemplary embodiments may provide a process for the synchronized management of personalized access data and access rights.

Another exemplary embodiment is a computer program with a program code for carrying out one of the processes being described here when the program code is executed on a computer, on a processor or on a programmable hardware component. A computer readable storage medium or machine-readable data storage medium, particularly a tangible medium excluding signals, carrier waves, or other transitory signals with such a program code stored thereon is another exemplary embodiment, namely a non-transitory, machine-readable tangible data storage media with program code stored thereon.

Exemplary embodiments create, moreover, a device for a communication component of a measuring system. The device comprises an interface, which is configured for communication in a network, and a control module, which is configured for carrying out one of the processes being described here.

Another exemplary embodiment is a communication component for a measuring system with such a device. The communication component may comprise one or more transmitters and/or one or more controllers.

Moreover, exemplary embodiments create a measuring system with a plurality of communication components as described here, wherein at least one first communication component comprises a controller and at least one second communication component comprises a transmitter. According to the above explanation, the measuring system may comprise a gas measuring system and/or a gas detection system.

Finally, exemplary embodiments also create a system with a plurality of communication components according to this description.

Some examples of devices and/or processes will be explained in more detail below with reference to the attached figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a block diagram of an exemplary embodiment of a process for a communication component of a measuring system;

FIG. 2 is a block diagram of an exemplary embodiment of a device for a communication component of a measuring system of an embodiment of a communication component and an exemplary embodiment of a measuring system;

FIG. 3 is a block diagram of an exemplary embodiment of a measuring system; and

FIG. 4 is a block diagram showing possible communication scenarios in an exemplary embodiment.

### DESCRIPTION OF PREFERRED EMBODIMENTS

Referring to the drawings, different examples will be described in more detail with reference to the attached figures. The thicknesses of lines, layers and/or areas may be exaggerated in the figures for illustration.

Further examples may cover modifications, equivalents and alternatives, which fall within the scope of the disclosure. Identical or similar reference numbers pertain in the entire description of the figures to identical or similar elements, which may be implemented identically or in a modified form in a comparison with one another, while they provide the same function or a similar function.

It is apparent that when an element is described as being connected” or “coupled” with another element, the elements may be connected or coupled directly or via one or more intermediate elements. When two elements A and B are combined with the use of an “or,” this shall be defined such that all possible combinations are disclosed, i.e., only A, only B as well as A and B, unless explicitly or implicitly defined otherwise. An alternative wording for the same combinations is “at least one of A and B” or “A and/or B.” The same holds true, mutatis mutandis, for combinations of more than two elements.

Increasing sensitivity to IT security leads to the need for measuring systems generally to authenticate the user, should the user want to access the data made available in terms of reading them (e.g., reading measured values or configuration data) or writing them (e.g., changing of configuration, calibration of sensors). The authentication can be performed, e.g., by the combination of username and a password. Based on the plurality of different analysis levels, this leads in conventional systems to a plurality of different access concepts and passwords for a technically related measuring chain. Exemplary embodiments make possible a simpler management of the access situation via this entire measuring chain.

The user management may be inconsistent in conventional systems, i.e., a user may have different access data at different components of the same measuring system, and this can lead to increased management efforts. Moreover, the user management may be unsynchronized, i.e., if a user should change user data, for example, a password, this will not lead to any changes on all other transmitters, controllers and possible applications in the cloud or in the local IT. Changes of the user data are then carried out possibly in a time-consuming manner on all individual devices. A user with global access rights, for example, an administrator, must then manage many conventional different passwords, even though an identical access code valid for the measuring system would be sufficient corresponding to his access authorization level.

Moreover, a user management is not personalized and there is, as a rule, only a role-based user management. A personalized withdrawal of right is thus ruled out. These points may especially impact security, because user data only can be handled globally with great efforts (e.g., rapid changes when needed); role-based passwords are known to all, and it is not possible at the user level to reconstruct who has made what changes (e.g., configuration) and at what time.

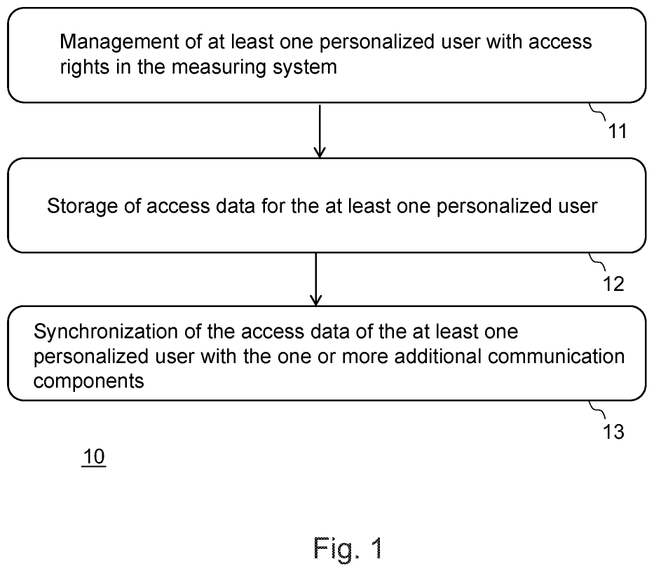

FIG. 1 shows a block diagram of an exemplary embodiment of a process 10 for a communication component of a measuring system. The measuring system has one or more additional communication components. The process 10 comprises a management 11 of at least one personalized user with access rights in the measuring system. Management is defined in this connection as the creation, definition, deletion, administration, specification, withdrawal, etc., of user data and access rights. Personalized users are those who are distinguished by individual access data from predefined user groups or roles, such as standard users, guest and administrator.

As is also shown in FIG. 1, the process 10 comprises a storage 12 of access data for the at least one personalized user and the access rights of that user. This may happen, for example, on a storage medium of any kind, examples being hard drives, network storage, main memories, etc. The process 10 further comprises a synchronization 13 of the access data of the at least one personalized user with access rights for the one or more additional communication components. Synchronization shall be defined here as a comparison of the access data such that the access data are identical at the components involved after the synchronization and they correspond, for example, to the most up-to-date version of the access data. The same holds true of the access rights. The communication component comprises a controller and the one or more additional communication components comprise one or more transmitters. The process 10 comprises the receipt of updated access data to the one or more transmitters or to another communication component communicating with the controller, and a standardization of the updated access data among the one or more additional communication components.

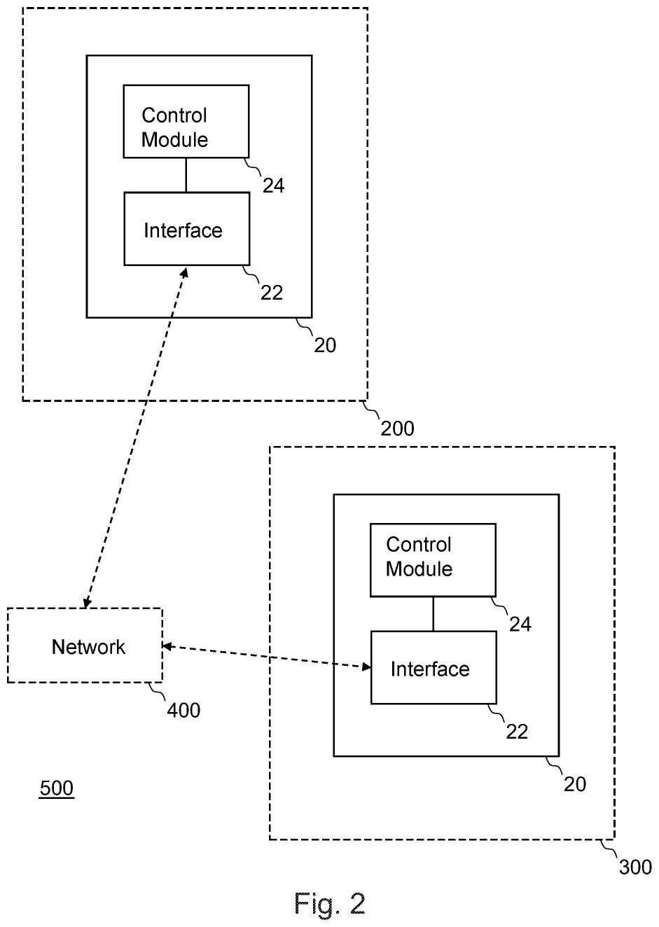

FIG. 2 shows a block diagram of an exemplary embodiment of a device 20 for a communication component 200, 300 of a measuring system 500. The device 20 for the communication component 200, 300 of the measuring system 500 comprises at least one interface 22, which is configured for the communication in a network 400. The device 20 further comprises a control module 24, which is coupled with the at least one interface 22 and which is configured to carry out one of the processes 10 being described here. FIG. 2 shows, moreover, two communication components 200, 300, which each comprise an exemplary embodiment of a device 20, and which are drawn in broken lines, because these shall be considered to be optional from the viewpoint of the device 20. Examples of such communication components 200, 300 are a controller, a transmitter or an access node for the network 400, for example, in a local component or also in the cloud. The network 400 may be, for example, an IP (internet protocol) network or a network based on a digital field bus. FIG. 2 shows, moreover, an exemplary embodiment of a measuring system 500, which comprises a plurality of communication components 200, 300 communicating via the network 400.

The interface 22 may be configured in exemplary embodiments as a typical interface for communication in networks 400. For example, this may be configured in exemplary embodiments by corresponding contacts. It may also be configured in exemplary embodiments as separate hardware and comprise a memory, which stores the signals to be transmitted and the signals received at least temporarily. The interface 22 may be configured to receive electrical signals, for example, as a bus interface, as an optical interface, as an Ethernet interface, as a wireless interface, as a field bus interface, as a HART interface, etc. It may, moreover, be configured in exemplary embodiments for wireless transmission and comprise a radio front end as well as corresponding antennas. Further, synchronization mechanisms for synchronization with the respective transmission medium for the one or more connection types may be comprised—synchronization mechanisms may be provided.

The control module 24 may comprise in exemplary embodiments one or more freely selectable controllers, microcontrollers, network processors, processor cores, such as Digital Signal Processor cores (DSPs), programmable hardware components, etc. Exemplary embodiments are not limited in this case to a defined type of processor core. Freely selectable processor cores or even a plurality of processor cores or microcontrollers may be provided for the implementation of a control module 24. The control module 24 may be implemented in an integrated form with other devices. For example, the control module 24 may be implemented in a control unit that additionally also comprises one or more other functions. A control module 24 may be embodied in exemplary embodiments by a processor core, by a computer processor core (CPU=Central Processing Unit), by a graphics processor core (GPU=Graphics Processing Unit), by an application-specific integrated circuit core (ASIC=Application Specific Integrated Circuit), by an integrated circuit (IC=Integrated Circuit), by a one-chip system core (SOC=System on Chip), by a programmable logic element or by a field-programmable gate array with a microprocessor (FPGA=Field Programmable Gate Array) as the core of the component or of the components.

Exemplary embodiments may generally be used in freely selectable measuring systems.

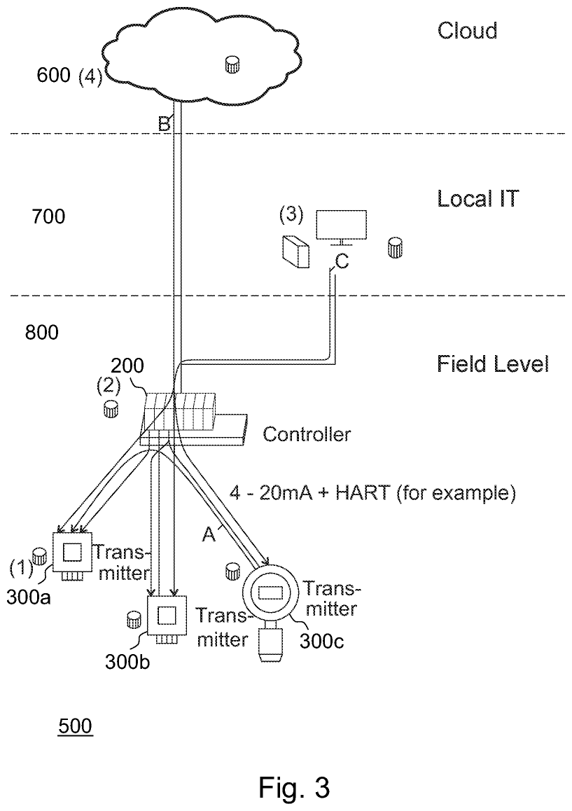

Especially the application in gas detection systems will be discussed below. Such gas detection systems comprise, as a rule, one or more sensors or transmitters, in conjunction with one or more analysis units (controllers) and optionally with the connection to a local IT (information technology) or cloud. The measuring system 500 accordingly comprises a gas measuring system and a gas detection system. FIG. 3 shows a block diagram of an exemplary embodiment of a measuring system 500. The detection function (switching of a countermeasure) is not explained concretely in FIG. 3. There may be for this purpose, starting from the controller 200, a connection to actuators, which generate, e.g., an optical or acoustic warning or an alarm.

The measuring system 500 is divided into three levels in this exemplary embodiment, namely, into a cloud level 600 at the top (in the sense of network cloud), a local IT level 700 in the center and a field level 800 at the bottom. A network cloud 600, which is composed, for example, of a plurality of computers, servers and/or access points arranged in a distributed manner in space, is located at the cloud level 600. For example, external access points may be implemented in this case for the measuring system 500.

For example, accesses by local computers, i.e., computers located at the same site with the measuring system 500, may be present at the local IT level 700. At least one controller 200 and three transmitters/sensors 300a, 300b and 300c are located at the field level 800 in this exemplary embodiment.

FIG. 3 illustrates communication components 200, 300a, 300b, 300c at the field level 800 as well as communication components, for example, computers or access nodes, at the levels 700 and 800 for the measuring system 500 with a respective device 20 each. The communication component 200 corresponds in this case to a controller and the communication components 300a, 300b and 300c correspond here to transmitters 300a, 300b and 300c. Thus, at least one first communication component 200 comprises a controller 200 and at least one second communication component 300a, 300b and 300c comprises a transmitter 300abc.

A sensor or transmitter 300a, 300b and 300c transmits the measurement of the gas concentration and forward this over, e.g., 4-20 mA in connection with a digital bus (e.g., HART, but also wireless) to the controller 200. Many variants are conceivable in exemplary embodiments concerning the connection. Examples are 4-20 mA alone (not digital in this case) or with HART, other communication protocols, such as Profibus, but also wireless protocols (operating, as a rule, without a 4-20 mA limitation). The controller 200 assumes the analysis of the signal and switches countermeasures (e.g., acoustic alarm signals) when a defined threshold is reached. The controller 200 also assumes the forwarding of the data, e.g., into a local IT 700 or cloud application 600, where further analyses or configurations can be carried out. The process 10 is then carried out in the measuring system 500 for the application of a standard user management for the measuring system 500. The synchronization 13 may then comprise a synchronization of the access data and access rights of the at least one personalized user. The access rights may be based on a role assigned to the at least one personalized user, for example, as a modified guest user.

As is shown in FIG. 3, the measuring system 500 comprises at least one controller 200 and one or more transmitters 300abc communicating digitally with the controller 200. The transmitters 300abc are coupled with gas measuring sensors in this case. The synchronization 13 is carried out in this case at least partially via a digital field bus between the controller 200 and the transmitters 300abc (e.g., 4-20 mA with HART). Moreover, at least some of the components involved can document a change of access data at all three levels 600, 700, 800.

For example, the controller 200 receives user data updated via an IP network for a defined user from a computer of the local IT 700. The synchronization 13 of the access data is then achieved by standardization of the access data at the plurality of transmitters 300abc by the controller 200. The updated access data and a standardization of the updated access data are thus carried out at the controller 200 and at one or more transmitters 300abc. The updated access data may also be received at other freely selectable access points of the measuring system 500 in other exemplary embodiments. For example, the updated access data may be received at the controller 200, at a transmitter 300abc or at another communication component communicating with the controller 200.

The other communication component communicating with the controller 200 may comprise, for example, a control room in an IT system 700, which is a local IT system for the measuring system, or an access node in a cloud 600. Measuring systems 500 usually have an administration (maintenance, management, technical support, etc.), such as a local IT 700, which is installed in the immediate local vicinity. It is, however, also conceivable that a measuring system is administered, maintained or covered by technical support, in addition or as an alternative, from a remotely located node. This may be a computer, which is connected via corresponding security mechanisms, e.g., encryption, VPN (Virtual Private Network), protocol tunnel, and which has access to the measuring system 500. It may be, in principle, a freely selectable access node in the cloud 600. The updated access data are standardized in this case at least in some exemplary embodiments at all communication components suitable for a user access at all levels 600, 700, 800. This may just as well pertain to the access rights, so that the process 10 also comprises a management and/or standardization of personalized access rights for the at least one personalized user.

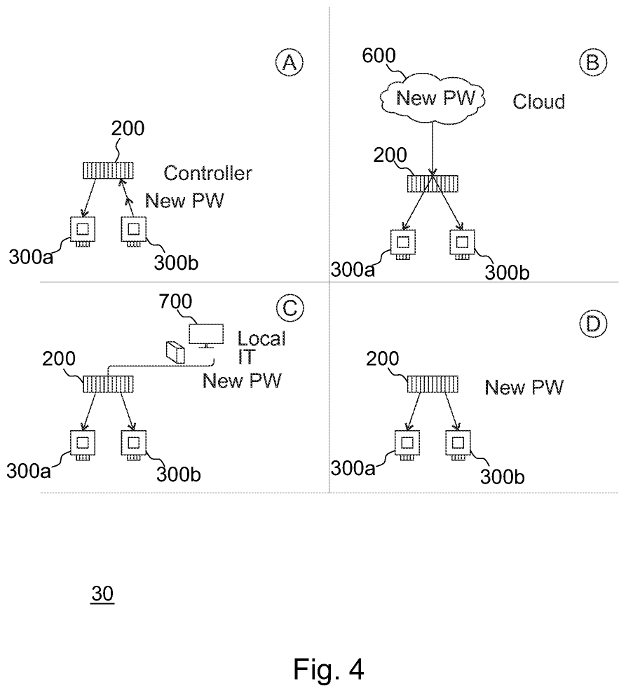

FIG. 4 shows possible communication scenarios in an exemplary embodiment. In a first scenario A, which is shown in the top left part of FIG. 4, the transmitter 300b receives a new password, which is communicated to the controller 200. The controller then passes the new password on to the transmitter 300a and it thus achieves a synchronization. For example, a field bus such as HART can be used as the communication connection. At the same time, the password may also be passed on to additional communication components at the levels above this.

Another scenario B is shown in the top right part of FIG. 4. A new password is assigned in this case at an access node or at a communication component in the cloud 600, and this password is then communicated to the controller 200 via the cloud. The controller 200 then passes the new password on to the transmitters 300a and 300b and thus the controller 200 synchronizes the transmitters 300a and 300b therewith.

FIG. 4 shows in the bottom left part a scenario C, in which a computer of the local IT 700 receives a new password and communicates this new password to the controller 200. The controller 200 then communicates the new password to the two transmitters 300a and 300b in order to achieve the synchronization of the access data.

In a scenario D, which is shown in the bottom right part of FIG. 4, the new password is entered directly at the controller 200 and the latter synchronizes the two transmitters 300a and 300b as well as optionally additional communication components at the levels 600 and 700.

A global user management for measuring systems 500 makes it possible to access the corresponding component from each access point (transmitter 300abc, controller 200, control room via local IT 700, cloud 800) concerning the same user data personalized for the user. The user data (user, role, password, etc.) may be stored for this purpose in an encrypted form at each access point. A synchronization is carried out in case of changes, restarts or in a time-controlled manner. This may be configured in further exemplary embodiments such that the user management relates to defined groups of individual components, for example, at all sensors and transmitters that serve the same measurement purpose in terms of measurement but are installed at different locations.

The following scenarios describe possible applications, cf. FIG. 4:
(A, top left): Two transmitters 300a, 300b with controller 200, no cloud: A new password is created for a user in the transmitter 300b; this password is also synchronized with the controller 200, and then with all other transmitters 300a;
(B, top right): Two transmitters 300a, 300b with controller 200 and cloud 600: A new user with password, role, etc., is created in the cloud 600. A synchronization is then carried out with the controller 200 and with the transmitters 300a, 300b;
(C, bottom left): Two transmitters 300a, 300b with controller 200 and application in local IT 700: A new user with password, role, etc., is created in the application. A synchronization is then carried out with the controller 200 and with the transmitters 300a, 300b;
(D, bottom right): Two transmitters 300a, 300b with controller 200, no cloud: A new password is created for a user in the controller 200, and a synchronization is then carried out with the transmitters 300a, 300b connected.
(E) (no image): Time- and location-limited authorizations for certain applications are possible. A time-limited authorization may comprise a limited-time access authorization for a user, e.g., for one minute, 5 minutes, 10 minutes, one hour, one day, etc. The time-limited authorization may also relate to exactly one access, for example, for one minute, 5 minutes, 10 minutes, one hour, etc. A location-limited authorization may pertain to a subgroup of the communication components of a system, for example, all transmitters/controllers in a partial area of the system, for example, along a defined line or in a defined process area.

In addition to time- or location-limited authorizations, functionally limited authorizations or authorization assignments may also occur in other exemplary embodiments. For example, provisions may be made in exemplary embodiments for authorizations to be assigned only for certain types of transmitters/sensors or communication components. This functional limitation may be useful in case of maintenance work, for example, when only defined types of maintenance are performed. Thus, different gas sensors, e.g., oxygen sensors and chlorine sensors, may be implemented, for example, in a gas measuring system in an exemplary embodiment. If maintenance shall only be performed on the oxygen sensors, it may happen that certain access rights necessary specifically for the oxygen sensors only shall be assigned. A standardization of the updated access data can thus be limited to the oxygen sensors.

The one or more communication components may thus also comprise transmitters or sensors with a defined functionality or of a certain type. The standardization of the updated access data among the one or more communication components may then comprise in some exemplary embodiment a standardization (unifying) of the access data among communication components in a defined three-dimensional space, in a management area (e.g., address space), for a defined time period and/or with a defined functionality.

A synchronization of the transmitters 300abc is carried out in the exemplary embodiments described so far by the controller 200. Other components or a transmitter itself may also coordinate or trigger the updating of additional transmitters in other exemplary embodiments. This may happen, for example, by broadcast messages to all components in a system, which can be sent, in principle, from each authorized access node or communication component.

Exemplary embodiments may, furthermore, be or pertain to a computer program with a program code for carrying out one or more of the above processes when the computer program is executed on a computer or processor. Steps, operations or processes of different processes described above may be executed by programmed computers or processors. Examples may also cover program storage devices, e.g., digital data storage media (non-transitory, machine-readable tangible data storage media), which are machine-readable, processor-readable or computer-readable and code machine-executable, processor-executable or computer-executable programs of instructions saved on tangible data storage media. The instructions execute some or all of the steps of the above-described processes or cause them to be executed. The program storage devices (tangible data storage media) may comprise or be, e.g., digital memories (flash memories or solid state drive memories), magnetic storage media, for example, magnetic disks and magnetic tapes, hard drives or optically readable digital data storage media—non-transitory, machine-readable tangible data storage media. Further examples may also cover computers, processors or control units, which are programmed for executing the steps of the above-described processes, or (field) programmable logic arrays ((F)PLAs=(Field) Programmable Logic Arrays) or (field)programmable gate arrays ((F)PGA=(Field) Programmable Gate Arrays), which are programmed for executing the steps of the above-described processes.

Functions of different elements shown in the figures as well as the designated function blocks may be implemented in the form of dedicated hardware, e.g., “of a signal provider,” etc., as well as, in the form of hardware capable of executing software in conjunction with corresponding software. In case of provision by a processor, the functions may be provided by an individual dedicated processor, by an individual, jointly used processor or by a plurality of individual processors, some of which or all of which may be used jointly. However, the term “processor” or “control” is far from being limited to hardware capable exclusively of executing software, but it may comprise digital processor hardware (DSP hardware; DSP=Digital Signal Processor), network processor, application-specific integrated circuit (ASIC=Application Specific Integrated Circuit), field-programmable logic array (FPGA=Field Programmable Gate Array), read-only memory (ROM=Read Only Memory) for storing software, random access memory (ROM=Random Access Memory) and non-volatile storage device (storage). Other hardware, conventional and/or customer-specific may be included as well.

A block diagram may represent, for example, a schematic circuit diagram, which implements the basic principles of the disclosure. Similarly, a flow chart, a flow diagram, a state transition diagram, a pseudocode and the like may represent different processes, operations or steps, which are represented, for example, essentially in computer-readable medium and are thus executed by a computer or processor, regardless of whether such a computer or processor is explicitly shown. Processes disclosed in the specification or in the patent claims may be implemented by a component, which has a means for executing each of the respective steps of these processes.

It is apparent that the disclosure of a plurality of steps, processes, operations or functions disclosed in the specification or in the claims shall not be interpreted as being in the defined order, unless this is explicitly or implicitly stated otherwise, e.g., for technical reasons. Therefore, these are not limited by the disclosure of a plurality of steps or functions to a defined order, unless these steps or functions are not replaceable for technical reasons. Further, an individual step, function, process or operation may include in some examples a plurality of partial steps, partial functions, partial processes or partial operations and/or be broken up into these. Such partial steps may be included and be part of the disclosure of this individual step, unless they are explicitly excluded.

While specific embodiments of the invention have been shown and described in detail to illustrate the application of the principles of the invention, it will be understood that the invention may be embodied otherwise without departing from such principles.

### LIST OF REFERENCE NUMBERS

10 Process for a communication component of a measuring system
11 Management of at least one personalized user with access rights in the measuring system
12 Storage of access data for the at least one personalized user
13 Synchronization of the access data of the at least one personalized user with the one or more additional communication components
20 Device for a communication component
22 Interface
24 Control module
200 Controller, communication component
300 Communication component
300a Transmitter, communication component
300b Transmitter, communication component
300c Transmitter, communication component
400 Network
500 Measuring system
600 Local IT, communication component
700 Cloud, communication component
800 Field level, communication component

What is claimed is:

1. A process for a communication component of a measuring system, wherein the measuring system has one or more additional communication components, the process comprising the steps of:
managing at least one personalized user with access rights to the measuring system;
storing access data for the at least one personalized user; and
synchronizing the access data of the at least one personalized user with the one or more additional communication components, wherein:
the communication component comprises a controller;
the one or more additional communication components comprises one or more transmitters;
the step of synchronizing comprises:
receiving updated access data at the one or more transmitters or at another communication component communicating with the controller; and
standardizing the updated access data among the one or more communication components.

2. A process in accordance with claim 1, wherein the measuring system comprises a gas measuring system.

3. A process in accordance with claim 1, wherein the strep of synchronizing further comprises a synchronization of the access rights of the at least one personalized user.

4. A process in accordance with claim 1, wherein the access rights are based on a role assigned to the at least one personalized user.

5. A process in accordance with claim 1, wherein the measuring system comprises the controller as at least one controller connected to or forming a part of the communication component and the one or more transmitters communicating digitally with the controller, wherein the transmitters are coupled with gas measuring sensors.

6. A process in accordance with claim 1, wherein the strep of synchronizing takes place at least partially via a digital field bus.

7. A process in accordance with claim 1, further comprising the step of documenting changes of the access data.

8. A process in accordance with claim 1, wherein the access rights of the user comprise one or more of time limitations and space limitations.

9. A process in accordance with claim 1, further comprising managing personalized access rights for the at least one personalized user.

10. A process in accordance with claim 1, further comprising providing a program code for carrying out at least one of the process steps, upon the program code being executed on a computer, on a processor, or on a programmable hardware component.

11. A device for a communication component of a measuring system, wherein the measuring system has one or more additional communication components, the device comprising:
an interface configured to communicate in a network; and
a control module configured:
to manage at least one personalized user with access rights to the measuring system;
to store access data for the at least one personalized user;
to synchronize the access data of the at least one personalized user with the one or more additional communication components, wherein:
the communication component comprises a controller;
the one or more additional communication components comprises one or more transmitters;
the control module synchronizes by:
sending updated access data to the one or more transmitters or to another communication component communicating with the controller; and
standardizing the updated access data among the one or more communication component.

12. A device according to claim 11 in combination with the one or more additional communication components.

13. A measuring system comprising:
at least one first communication component comprising a controller and an interface configured to communicate in a network; and
at least one second communication component comprising a transmitter, wherein the controller is configured:
to manage at least one personalized user with access rights to the measuring system;
to store access data for the at least one personalized user;
to synchronize the access data of the at least one personalized user with the at least one second communication component, wherein the controller control module synchronizes by:
sending updated access data to the one or more transmitters or to another communication component communicating with the controller; and
standardizing the updated access data among the one or more communication component.

14. A measuring system in accordance with claim 13, wherein the system comprises a gas measuring system.

15. A non-transitory, machine-readable, tangible data storage medium having stored thereon a computer program with a program code for carrying out one or more steps of a process for a communication component of a measuring system upon executing the program code on a computer, on a processor or on a programmable hardware component, wherein the measuring system has one or more additional communication components, the process comprising the steps of:
managing at least one personalized user with access rights to the measuring system;
storing access data for the at least one personalized user; and
synchronizing the access data of the at least one personalized user with the one or more additional communication components, wherein:
the communication component comprises a controller;
the one or more additional communication components comprises one or more transmitters;
the step of synchronizing comprises:
sending updated access data to the one or more transmitters or to another communication component communicating with the controller; and
standardizing the updated access data among the one or more communication components.
